(19) United States
(12) Patent Application Publication
(10) Pub. No.: US 2016/0038530 A1
(43) Pub. Date: Feb. 11, 2016

(54) Nutritional Supplement to Complement Cancer Therapy

(71) Applicant: Kevin Engholdt, Scottsdale, AZ (US)

(72) Inventor: Kevin Engholdt, Scottsdale, AZ (US)

(21) Appl. No.: 14/454,548

(22) Filed: Aug. 7, 2014

(51) Int. Cl. A61K 31/737; A61K 31/015; A61K 31/375; A61K 31/593; A61K 31/355; A61K 33/10; A61K 31/525; A61K 31/455; A61K 31/675; A61K 31/519; A61K 31/714; A61K 31/197; A61K 31/716; A61K 31/51

(52) U.S. Cl. CPC A61K 31/737; A61K 31/716; A61K 31/015; A61K 31/375; A61K 31/593; A61K 31/355; A61K 31/51; A61K 31/525; A61K 31/455; A61K 31/675; A61K 31/519; A61K 31/714; A61K 31/197; A61K 33/10

(57) ABSTRACT

A nutritional or dietary supplement composition that strengthens and promotes health through the prevention, stabilization, reversal and/or treatment of cancers, chemotherapy, and/or radiation therapy related complications. The nutritional or dietary supplement composition may likewise reduce the risk and/or prevalence of cancerous cells in addition to providing complement to cancer related therapies. The essential ingredients of the nutritional or dietary supplement composition are vitamin A, vitamin B6, vitamin B12, vitamin C, vitamin D3, vitamin E, calcium, thiamin, riboflavin, niacin, folic acid, pantothenic acid, fucoidan (extract from seaweed species such as, but not limited to; laminaria japonica and undaria pinnatifida), beta-glucans (extract from mushroom species such as, but not limited to; ganoderma lucidum and grifola frondosa), and antioxidants (from vegetable, fruit, or herb such as, but not limited to; acai berry (Euterpe oleracea), sumac, cloves, and sorghum). The essential ingredients are preferably provided in a capsule or tablet form suitable for oral ingestion. Preferably the composition is taken in the form of 3 tablets taken once daily.

### FIELD OF INVENTION

The present invention relates to a nutritional or dietary supplement composition that strengthens and promotes immune health through the prevention, stabilization, reversal and/or treatment of cancerous cells. More specifically, the present invention will aid in complementing traditional cancer therapies as it is well tolerated with no toxicity and aids in reduction of cancer cell growth in a synergistic fashion of its inherent comprehensive blend with antioxidant capabilities to aid in cellular longevity, apoptotic properties for cancerous-only cells, and immuno-stimulating properties.

### BACKGROUND OF INVENTION

Cancer is the leading cause of death worldwide. Cancer is a generic term for a large group of diseases that can affect any part of the body. Other terms used are malignant tumors and neoplasms. One defining feature of cancer is the rapid creation of abnormal cells that grow beyond their usual boundaries, and which can then invade adjoining parts of the body and spread to other organs. This process is referred to as metastasis. Metastases are the major cause of death from cancer. Cancer is a leading cause of death worldwide, accounting for 8.2 million deaths in 2012 (1). The main types of cancer are:
lung (1.59 million deaths)
liver (745 000 deaths)
stomach (723 000 deaths)
colorectal (694 000 deaths)
breast (521 000 deaths)
oesophageal cancer (400 000 deaths) (1).

Cancer arises from one single cell. The transformation from a normal cell into a tumor cell is a multistage process, typically a progression from a pre-cancerous lesion to malignant tumors. These changes are the result of the interaction between a person's genetic factors and three categories of external agents, including:
physical carcinogens, such as ultraviolet and ionizing radiation;
chemical carcinogens, such as asbestos, components of tobacco smoke, aflatoxin (a food contaminant) and arsenic (a drinking water contaminant); and
biological carcinogens, such as infections from certain viruses, bacteria or parasites.

Recently, attention has been focused on using natural supplements to help prevent and reduce the onset of cancerous cells and maintain and stimulate immune response. Two specific extracts have received particular notice, fucoidan (from seaweed) and beta-glucans (from mushrooms). These two specific extracts, by themselves, have shown clinical evidence of reducing cancerous cells and immune balancing properties. This art is recognizing the combination of these two extracts (including but not limited to seaweed species such as laminaria japonica and undaria pinnatifida and mushroom species ganoderma lucidum and grifola frondosa) with antioxidant fruits, vegetable or herbs (including but not limited to acai berry (Euterpe oleracea), Sumac, and cloves) and a ten essential multivitamins and minerals (vitamin A, vitamin B6, vitamin B12, vitamin C, vitamin D3, vitamin E, calcium, thiamin, riboflavin, niacin, folic acid, pantothenic acid) and their synergistic properties to prevent and/or reduce the onset of cancerous cells and maintain and stimulate immune response.

Vitamins B6, B12, C, E and Folic Acid are well known and documented to aid in heart health, vitamins A, C, and E for immunity and eye health, and finally, Thiamin, Riboflavin and Niacin to aid in converting food to fuel and maintain physical energy.

Antioxidants; fruit, vegetable or herb (including but not limited to Acai berry [(Euterpe oleracea), Sumac, and cloves)] have gained increased clinical evidence as being a strong antioxidant for overall cell structure and reducing toxicity from free radicals and oxidizers promoting cell longevity.

Beta-glucans—These compounds have shown dramatic immunostimulating as well as immune balancing properties.

Fucoidan—These compounds have been shown to have pro-apototic effects on cancerous cells as well as promoting immune response to foreign bodies.

Currently, there is no nutritional complement to patients undergoing cancer therapies, moreover, no nutritional complement that may aid them in toleration as well as recovery during that time period. Accordingly, a need exists in the to provide methods and compositions for the complementary treatment of cancer related treatments.

### SUMMARY OF INVENTION

The present invention is a nutritional or dietary supplement composition for administration to humans or other animals that strengthens and promotes cell health through the prevention, stabilization, reversal and/or treatment of cancer in complement to traditional cancer therapies for persons with particular diseases. The present nutritional or dietary supplement composition preferably comprises an effective and efficient amount of specific antioxidants, immune stimulating, immune balancing, antitumor properties with an essential base vitamin and mineral panel to aid in toleration and recovery of cancer therapies. The practice of this invention involves supplementing the diet of humans or animals by oral, intraperitoneal, intravenous, subcutaneous, transcutaneous or intramuscular routes of administration.

Another object of the present invention is to provide a safe nutritional or dietary supplement composition that strengthens and promotes cell health through the prevention, stabilization, reversal and/or treatment of cancer in complement to traditional cancer therapies for persons with particular diseases.

### DETAILED DESCRIPTION

The following detailed description is provided to enable any person skilled in the art to which the present invention pertains to make and use the same, and sets forth the best mode contemplated by the inventors of carrying out the subject invention.

The preferred nutritional or dietary supplement composition of the present invention is a formulation of five essential ingredients preferably in quantities not less than those set forth below in Table 1, take as 3 tablets, to be ingested daily.

TABLE 1

| Composition | Amount |
|---|---|
| Vitamin A (28% as beta carotene) | 5000 International Units (IU) |
| Vitamin C (as ascorbic acid) | 60 mg |
| Vitamin D3 (as cholecalciferol) | 400 International Units (IU) |
| Vitamin E (as acetate) | 30 International Units (IU) |
| Thiamin (B1) | 1.5 mg |
| Riboflavin (B2) | 1.7 mg |
| Niacin | 20 mg |
| Vitamin B6 (as pyroxidine HCL) | 2 mg |
| Folic Acid | 400 mcg |
| Vitamin B12 (as cyanocobalamin) | 6 mcg |
| Pantothenic Acid (as d-Calcium | 10 mg |
| Pantothanate) | |
| Calcium (as calcium carbonate) | 45 mg |
| (Fucoidan) Laminaria japonica | 350 mg |
| (Fucoidan) Undaria pinnatifida | 300 mg |
| (Beta Glucan) Grifola frondosa | 300 mg |
| (Beta Glucan) Ganoderma lucidum | 300 mg |
| (Antioxidant) Euterpe oleracea | 200 mg |

The subject composition is formulated to provide the above-listed essential ingredients at preferably not less than the daily dosage amounts specified above. As stated above, the species of seaweed, mushroom, and antioxidant are specifically used in this formulation due to their research evidence but are not limited to only these species. Any seaweed and/or mushroom and/or antioxidant fruit, vegetable or herb species is considered part of this formulation and thereby synergistic effect within the combination. The subject composition is preferably provided for oral administration in the form of lacquered tablets, unlacquered tablets, caplets or capsules. For purposes of simplicity only, throughout the remainder of this detailed description lacquered tablets, unlacquered tablets, caplets and capsules will each be referred to as simply “tablets” without distinction in form or function there between. Additionally, for purposes of simplicity only, throughout the remainder of the detailed description, all species of seaweed described will be referred to as simply “seaweed” as well as all species mushrooms described will be referred to as simply as “mushroom”. Finally, for purposes of simplicity only, throughout the remainder of the detailed description, the antioxidant set of formulations including but not limited to acai, sumac, and cloves will simply be “antioxidants” for remainder of this detailed description.

The preferred daily dosage of the subject composition as specified above may be administered in the form of three or more tablets. Most preferably the daily dosage of the subject composition is provided in the form of three tablets taken once daily. The three capsules are preferably taken at the same time to provide improved absorption and better maintenance of blood levels of the essential ingredients as shown from the evidence of their effect. The minimum quantities specified above, per tablet, reflect the minimum amount of each essential ingredient to be provided upon oral administration through to the date of tablet expiration as set forth on the tablet sale label. Typically, the product shelf life for nutritional or dietary supplements is approximately two to three years.

Variations contemplated in administering the subject composition to humans or other animals include, but are not limited to, providing time-release tablets or tablets manufactured to be administered as a single dose or as other multiple part dosages. Additionally, alternative avenues of administration besides oral administration are contemplated herein such as for example, but not limited to, intraperitoneal, intravenous, subcutaneous, sublingual, transcutaneous, intramuscular or like forms of administration. Each tablet of the subject composition preferably contains the following essential ingredients in the quantities specified below. For purposes of simplicity only, formulations of the subject composition are provided below in accordance with a three-tablet oral daily dosage regime as described above.

### Vitamin A

Beta-carotene, a proform of vitamin A, is a lipid-soluble orange pigment found in many vegetables. Beta-carotene is converted to vitamin A in the body with an efficiency of approximately 50 percent. The US daily recommended allowance (RDA) of vitamin A is 5,000 IU. Beta-carotene has one of the highest antioxidant potentials of the antioxidants. No observed adverse effects are observed for beta-carotene at dosage levels as high as 25 mg per day for healthy, non-smokers. Such a formulation provides a total daily dosage of beta-carotene and Vitamin A. Beta-carotene is preferred in the subject composition due to its ready commercial availability although alternative carotenoid proforms of vitamin A could likewise be used.

### Vitamin C

Vitamin C is a well known water-soluble antioxidant. Humans depend on external sources of vitamin C to meet their vitamin C requirements.

The U.S. recommended dietary allowance (RDA) for vitamin C in the form of ascorbic acid is 60 mg. Very large daily doses of vitamin C have been taken over many years with no or only minor undesirable effects. Intakes of 1,000 mg or more of vitamin C can be consumed daily without any known adverse effects. The subject composition provides a daily dose of vitamin C.

Vitamin C is also well known to aid in the bioavailability as well as absorption of large macro molecules and proteins. Specifically, Vitamin C has been shown to aid in absorption of beta-glucans from mushrooms such as Ganoderm lucidum and Grifola frondosa rendering the inherent immunostimulant properties from these mushrooms more available into the bloodstream. In addition, the combination of Vitamin C and Fucoidan from seaweed species such as Laminaria Japonica and Undaria Pinnatifida have shown enhanced antioxidant capabilities and anti-tumor properties via inhibition of tumor invasion of human fibrosarcoma HT-1080 cells. Ascorbic acid is the preferred source of vitamin C in the subject tablets, although other sources such as for example sodium ascorbate could alternatively be used.

### Vitamin D3

Vitamin D is found in many foods, including fish, eggs, fortified milk, and cod liver oil. The sun (UV light) is the catalyst for the body to produce vitamin D. The term “vitamin D” refers to several different forms of this vitamin. Two forms are important in humans: vitamin D2, which is made by plants, and vitamin D3, which is made by human skin when exposed to sunlight. The said formulation is using D3 at the US recommended daily allowance of 400 International Units (IU) but said formulations may also use Vitamin D2. Vitamin D3 is not readily available to all populations and is in said formulation due to this reason.

The major role of vitamin D is to maintain normal blood levels of calcium and phosphorus. Vitamin D helps the body absorb calcium, which forms and maintains strong bones. It is used alone or together with calcium to improve bone health and decrease fractures.

### Vitamin E

Vitamin E is also a well-known antioxidant. Vitamin E can work synergistically with vitamin C in protecting vital cell function from normal oxidants. Vitamin E is a relatively non-toxic fat-soluble vitamin. Vitamin E is readily oxidized thereby significantly reducing its activity during periods of storage prior to ingestion. Once ingested, vitamin E is stored 45 within the body and can contribute to the total body pool of vitamin E for up to one year.

The RDA of vitamin E in the form of dl-alpha tocopheryl acetate is 30 IU which is in the said formulation. No adverse effects of dl-alpha tocopheryl acetate have been observed at levels as high as 800 mg, with 1.0 mg of dl-alpha tocopheryl acetate being equal to 1 IU of dl-alpha tocopheryl acetate. Dl-alpha tocopheryl acetate is the preferred source of vitamin E in the subject tablets although other sources of vitamin E, such as for example trimethyl tocopheryl acetate and/or vitamin E succinate, may be used in the alternative.

### Thiamin (B1)

Thiamine is involved in many body functions, including nervous system and muscle function, the flow of electrolytes in and out of nerve and muscle cells, digestion, and carbohydrate metabolism. Very little thiamine is stored in the body and depletion can occur within 14 days. Severe thiamine deficiency may lead to serious complications involving the nervous system, brain, muscles, heart, and stomach and intestines. The RDA for Thiamin is 1.5 mg and is within said formulation.

### Riboflavin (B2)

Riboflavin is a B vitamin. It can be found in certain foods such as milk, meat, eggs, nuts, enriched flour, and green vegetables. Riboflavin is frequently used in combination with other B vitamins in vitamin B complex products. Riboflavin is well known to increase energy levels; boosting immune system function; maintain healthy hair, skin, mucous membranes, nails, while slowing aging; boosting athletic performance and promoting healthy reproductive function. The RDA for Riboflavin is 1.7 mg and is within said formulation.

### Niacin (Vitamin B3)

Niacin is well known as required for the proper function of fats and sugars in the body and to maintain healthy cells. Niacin and nicinamide are forms of Vitamin B3. Vitamin B3 is found in many foods including yeast, meat, fish, milk, eggs, green vegetables, beans, and cereal grains. The RDA for Niacin is 20 mg and is within said formulation.

### Vitamin B6

Vitamin B6 is a vitamin that is naturally present in many foods. The body needs vitamin B6 for more than 100 enzyme reactions involved in metabolism. Vitamin B6 is also involved in as immune function. It is well know that people with low levels of vitamin B6 in the blood may have a higher risk of certain kinds of cancer, such as colorectal cancer.

### Folic Acid (Vitamin B9)

Folic Acid is well known to be necessary for strong blood. Lack of folic acid may lead to anemia (weak blood). Folic acid ability to maintain strong blood is well known to be enhanced when used along with vitamin B12. The RDA for Folic Acid is 400 mcg and is within said formulation.

### Vitamin B12

Vitamin B 12 is a nutrient that helps keep the body's nerve and blood cells healthy and helps make DNA, the genetic material in all cells. Vitamin B12 also helps prevent a type of anemia called megaloblasic anemia that makes people tired and weak. The current RDA for Vitamin B12 is 6 mcg and is within said formulation.

### Pantothenic Acid (Vitamin B5)

Pantothenic Acid, also known as Vitamin B5 essential to all life and is a component of coenzyme A (CoA), a molecule that is necessary for numerous vital chemical reactions to occur in cells. Pantothenic acid is essential to the metabolism of carbohydrates, proteins, and fats, as well as for the synthesis of hormones and cholesterol. The RDA is 10 mg, and is within said formulation.

This formulation can also be in form as D-pantothenic acid, as well as dexpanthenol and calcium pantothenate, which are chemicals made in the lab from D-pantothenic acid.

### Calcium

Calcium is required for vascular contraction and vasodilation, muscle function, nerve transmission, intracellular signaling and hormonal secretion, though less than 1% of total body calcium is needed to support these critical metabolic functions. Serum calcium is very tightly regulated and does not fluctuate with changes in dietary intakes; the body uses bone tissue as a reservoir for, and source of calcium, to maintain constant concentrations of calcium in blood, muscle, and intercellular fluids. The RDA for calcium is 900 mg, and this formulation may have anywhere from 1-100% of the calcium needs.

### Seaweed

Seaweed has been a staple food for many Asian countries with no toxicity of note. An extract of seaweed, fucoidan, has been widely shown to have multiple immune stimulating and anti-cancer properties. Seaweed species, including but not limited to, laminaria japonica and undaria pinnatifida, have shown the highest clinical potential for immune stimulation and anti-cancer properties. Alone, they have been shown to help mitigate post chemotherapy and radiation therapy complications for patients with cancers. The formulation has 300 mg of each species listed and is considered the minimal amount needed for a clinical effect. Again, not limited to these two species of seaweed, but have the highest effect from their fucoidans.

### Mushroom

Mushrooms have been a staple medicinal and food product for Asian countries with no toxicity of note. An extract of mushrooms, beta-glucans, has been widely shown to have immune modulating properties as well as anti-cancer properties. Mushroom species, including but not limited to, ganoderma lucidum and grifola frondosa, have shown significant clinical effect for immune modulation and anti-cancer properties. Alone, they have been shown to help mitigate post chemotherapy and radiation therapy complications for patients with cancers. The formulation has 300 mg of each species listed and is considered the minimal amount needed for clinical effect. Again, not limited to these two species of mushroom, but have the highest effect from their beta-glucans.

### Antioxidant (fruit, vegetable, and/or herb)

Antioxidants have been shown to extend the life of cells by reducing telomeric degradation. A unit of measure for antioxidant capacity is the oxygen radical absorbance capacity (ORAC). For purposes of this invention, “antioxidants” use for the formulation are any of the documented ORAC values exceeding 100,000 or more. Fruits, vegetables, and herbs including, but not limited to, such as Euterpe oleracea (Acai berry), sumac, cloves, and cinnamon are considered the highest known antioxidants as measured by the ORAC. The antioxidant in said formulation is 200 mg as this has been determined the minimal amount needed for clinical effect on healthy cells.

### REFERENCES

1. Attached to patent application

We claim:

1. A immune strengthening composition to complement cancer therapy comprising on a daily dosage basis:
a. 100% of RDA of Vitamins A, C, D3, E, Thiamin, Riboflavin, Niacin, Vitamin B6, Folic Acid, Pantothenic Acid
b. 5% of RDA of Calcium
c. Equal to or greater than 300 mg of Fucoidan from seaweeds
d. Equal to or greater than 300 mg of Beta-glucans from mushrooms
e. Equal to or greater than 200 mg of Antioxidants from fruit, vegetable, and/or herb that are documented greater than or equal to 100,000 ORAC unit of measure.s

2. A composition of claim 1 wherein provided in the form of capsules or tablets.

3. A composition of claim 1 or 2 wherein is taken orally 1 time daily.

4. A composition of claim 1 or 2 or 3 wherein taken in the form of 3 capsules or tablets each day.
